(19) United States
(12) Patent Application Publication
(10) Pub. No.: US 2026/0083463 A1
(43) Pub. Date: Mar. 26, 2026

(54) HOOK PLATE GUIDE AND METHOD OF USE

(71) Applicant: Skeletal Dynamics, Inc., Miami, FL (US)

(72) Inventors: Jorge L. ORBAY, Miami, FL (US); Yoelmir SANTANA, Miami, FL (US); Victor Morales, Miami, FL (US)

(21) Appl. No.: 18/896,072

(22) Filed: Sep. 25, 2024

(51) Int. Cl. A61B 17/17; A61B 17/80; A61B 17/90

(52) U.S. Cl. CPC A61B 17/1728; A61B 17/808; A61B 17/90

(57) ABSTRACT

A hook plate guide and method of use to facilitate engagement of a hook plate to a fracture fixation plate and to at least one bone fragment in a fractured bone; the guide comprising a proximal end, a distal end having a head, and a substantially elongated rigid body with a slot adapted to receive a hook plate guide fastener and adapted to slidably secure the hook plate guide to the fracture fixation plate; the head extending outward and having at least one reference hole and at least one drill hole adapted to be positioned over the at least one bone fragment; each at least one drill hole adjacent to a respective at least one reference hole.

### TECHNICAL FIELD

The technical field relates generally to the fixation of bone fractures, and in particular to guides for facilitating the placement of plates, such as hook plates, used in the fixation of bone fractures.

### BACKGROUND

Surgical plates for the fixation of bone fractures are known in the art. Such plates are primarily used to reduce and secure bone fragments that are caused by trauma, degeneration, or some other condition in the bone being repaired. On occasion, a single base plate is not sufficient to reduce and secure all of the bone fragments in a fractured bone because such additional bone fragments (i.e., “marginal fragments”) are beyond the reach of coverage of the base plate. In those situations, a second plate can be attached to the base plate, and the second plate can be extended from the base plate to reduce and secure these additional bone fragments.

One example of such an arrangement is disclosed in U.S. Pat. No. 10,603,090 which describes the combination of a volar fracture fixation plate and a hook plate, and is incorporated herein by reference. The hook plate is adapted to removably and adjustably attach to the volar plate. Moreover, the hook plate is equipped with a portion which wraps around the volar plate and includes at least two projections that extend beyond the volar plate and are adapted to reduce and secure any marginal bone fragments that cannot be directly reduced or secured by the volar plate.

Although this arrangement is very effective once in place, it can be difficult, due to, among other reasons, the small size and random shape of the marginal fragments, to reduce and secure the marginal fragments while installing the hook plate. In particular, it can be very difficult to simultaneously and with precision maintain the alignment of the marginal fragments once reduced, secure the hook plate projections to the fragments, and attach the hook plate to the volar plate.

Accordingly, there is presently a need in the art for an apparatus and method to facilitate with substantial precision (a) movable attachment of a hook plate onto a base plate; (b) positive engagement of one or more marginal fragments by the hook plate; and (c) securement of the hook plate to the base plate once the desired positioning of the bone fragments is achieved.

### SUMMARY

It is among the objects of the present apparatus and method of use to overcome the limitations of the heretofore-known devices by providing inventive features to achieve the precise and relatively less difficult reduction and securing of marginal bone fragments in a bone fracture. These objects are provided through a guide, and a method of use for the guide, that facilitates, without limitation, (a) precise movable attachment of a hook plate onto a base plate; (b) precise engagement of one or more marginal fragments by the hook plate; (c) more reliable alignment of reduced bone fragments; and (d) precise securement of the hook plate to the base plate once the desired positioning of one or more bone fragments is achieved.

It is an object of the present apparatus to provide a hook plate guide for facilitating engagement of a hook plate to a fracture fixation plate and to at least one bone fragment of a fractured bone. The hook plate may have at least one hook end for securing the at least one bone fragment. In some embodiments, the hook plate may have at least two hook ends separated by a distance. In at least one embodiment, the hook plate guide comprises a distal end, a proximal end, and a substantially elongated rigid body therebetween.

The body optionally comprises a slot. The slot may be elongated and adapted to receive a hook plate guide fastener. The slot is further adapted to slidably secure the hook plate guide to the fracture fixation plate.

The distal end comprises a head. The head further comprises at least one reference hole and at least one drill hole. In some embodiments, the head may comprise at least two reference holes and at least two drill holes with a distance between each of the at least two drill holes. The head may extend outward. The at least one reference hole and the at least one drill hole may be adapted to be positioned over the at least one bone fragment. In at least one embodiment, each at least one drill hole is adjacent to a respective at least one reference hole. In some embodiments, a position of each at least one drill hole corresponds to a position of a respective at least one hook end of the hook plate. In embodiments with at least two reference holes and at least two drill holes, the distance between each of the at least two drill holes may correspond to a distance between each of at least two hook ends of the hook plate.

In some embodiments, each at least one reference hole may be adapted to receive a Kirschner wire (k-wire). In further embodiments, each at least one drill hole may be adapted to receive a drill.

In at least one embodiment, the head may optionally be connected to the body by an arm. In at least one further embodiment, the arm may extend distally and curve downward to a position below the body of the hook plate guide.

In another embodiment, the head may be elongated and intersect the body at an angle that is non-perpendicular. In embodiments having an arm, the head may be elongated and intersect the arm at an angle that is non-perpendicular. In yet another embodiment, the head may optionally further comprise a spike adapted to engage the at least one bone fragment.

In other embodiments, the proximal end of the hook plate guide may further comprise a grip member extending proximally from the body to a position above the body. In such embodiments, the grip member may be adapted to facilitate in positioning the hook plate guide.

It is an object of the present method to provide a method of using a hook plate guide to facilitate engagement of a hook plate to a fracture fixation plate and to at least one bone fragment in a fractured bone. The fracture fixation plate comprises a bone contacting surface. The hook plate comprises at least one hook end. In at least one embodiment, the hook plate comprises at least two hook ends.

In at least one embodiment, the method comprises rigidly engaging the bone contacting surface of the fracture fixation plate to the fractured bone. Next, the at least one bone fragment is moved into a reduced position with respect to the fractured bone. The hook plate guide is then superimposed onto the fracture fixation plate. The hook plate guide may comprise a distal end having a head, a proximal end, and a substantially rigid body therebetween. In some embodiments, the body of the hook plate guide may optionally further comprise an elongated slot adapted to slidably secure the hook plate guide to the fracture fixation plate using a hook plate guide fastener. The head may have at least one reference hole and at least one drill hole adapted to be positioned over the at least one bone fragment. In at least some embodiments, the head comprises at least two reference holes and at least two drill holes adapted to be positioned over the at least one bone fragment.

The at least one reference hole and the at least one drill hole are then aligned with the at least one bone fragment. Thereafter, the hook plate guide may be temporarily affixed to the fracture fixation plate.

Optionally, the head of the hook plate guide may further comprise a spike. In such embodiments having a spike on the head of the hook plate guide, the spike may optionally engage the at least one bone fragment prior to the step of temporarily affixing the hook plate guide to the fracture fixation plate.

A k-wire is then inserted through each at least one reference hole of the hook plate guide and into the at least one bone fragment. The k-wire is adapted to hold the at least one bone fragment in the reduced position.

Next, at least one bone fragment hole is formed by drilling through each at least one drill hole of the hook plate guide and into the at least one bone fragment. In embodiments having at least two drill holes, at least two bone fragment holes are formed when drilling through each drill hole. The hook plate guide is then removed from the fracture fixation plate without displacing each k-wire inserted in the at least one bone fragment.

Thereafter, each at least one hook end of the hook plate is inserted into a respective at least one bone fragment hole. The hook plate may then be permanently affixed to the fracture fixation plate. Finally, each k-wire is removed from the at least one bone fragment.

Although the apparatus and method of use are illustrated and described herein as embodied in a guide for facilitating engagement of a hook plate to a volar fracture fixation plate for the distal radius, it is nevertheless not intended to be limited to only the details shown, since various modifications and structural changes may be made therein without departing from the spirit or purview of the application and within the scope and range of equivalents of the claims.

The construction of the apparatus and method of use, however, together with additional objects and advantages thereof will be best understood from the following description of the specific disclosed embodiments when read in connection with the accompanying drawings.

### BRIEF DESCRIPTION OF THE DRAWINGS

FIG. 1 illustrates a perspective view of a hook plate guide in accordance with one embodiment.

FIGS. 2 and 3 illustrate top and bottom views, respectively, of the hook plate guide of FIG. 1 in accordance with one embodiment.

FIG. 4 illustrates a side view of the hook plate guide of FIG. 1 in accordance with one embodiment.

FIG. 5 illustrates a perspective view of a hook plate guide having a head with a spike in accordance with another embodiment.

FIGS. 6 and 7 illustrate top and bottom views, respectively, of the hook plate guide of FIG. 5 in accordance with one embodiment.

FIG. 8 illustrates a side view of the hook plate guide of FIG. 5 in accordance with one embodiment.

FIG. 9 illustrates a perspective view of a hook plate guide in accordance with another embodiment.

FIGS. 10 and 11 illustrate top and bottom views, respectively, of the hook plate guide of FIG. 9 in accordance with one embodiment.

FIG. 12 illustrates a side view of the hook plate guide of FIG. 9 in accordance with one embodiment.

FIG. 13 illustrates a perspective view of a fastener.

FIGS. 14 and 15 illustrate top and bottom views, respectively, of a fracture fixation plate.

FIG. 16 illustrates an enlarged perspective view of the fracture fixation plate of FIGS. 14 and 15 showing additional details.

FIG. 17 illustrates a perspective view of a hook plate.

FIG. 18 illustrates a perspective view of a hook plate having an elongated tab.

FIG. 19 illustrates a fracture fixation plate engaged with a fractured bone having at least one bone fragment in accordance with one embodiment.

FIG. 20 illustrates a hook plate guide temporarily affixed to a fracture fixation plate in accordance with one embodiment.

FIG. 21 illustrates a k-wire inserted through each at least one reference hole of a hook plate guide and into at least one bone fragment of a fractured bone in accordance with one embodiment.

FIG. 22 illustrates k-wires inserted into at least one bone fragment of a fractured bone after a hook plate guide has been removed in accordance with one embodiment.

FIG. 23 illustrates a hook plate affixed to a fracture fixation plate and each at least one hook end of the hook plate inserted into a respective at least one bone fragment hole in accordance with one embodiment.

FIG. 24 illustrates a hook plate affixed to a fracture fixation plate and at least one bone fragment of a fractured bone in accordance with one embodiment.

FIG. 25 illustrates a listing of steps according to a method of using a hook plate guide to facilitate engagement of a hook plate to a fracture fixation plate and to at least one bone fragment in a fractured bone in accordance with one embodiment.

### DETAILED DESCRIPTION

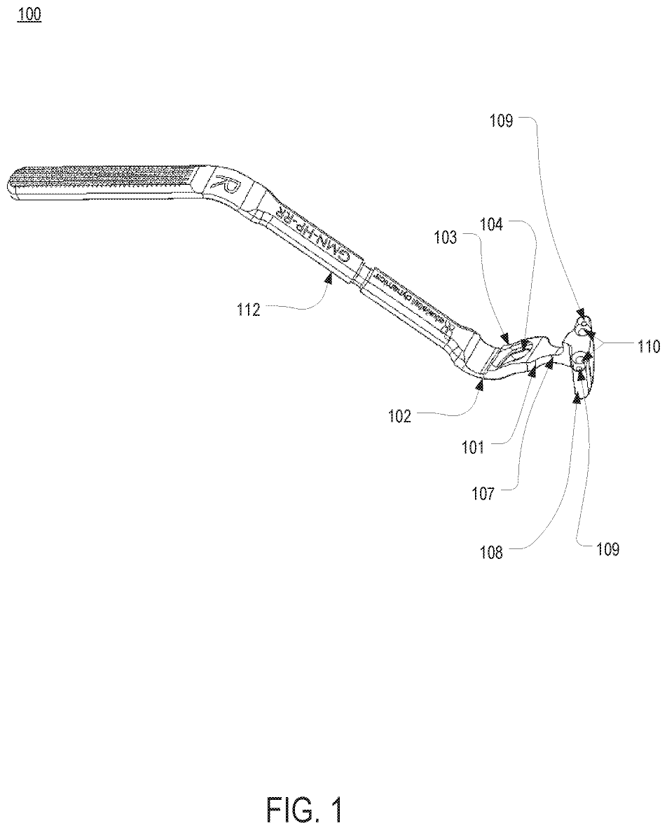

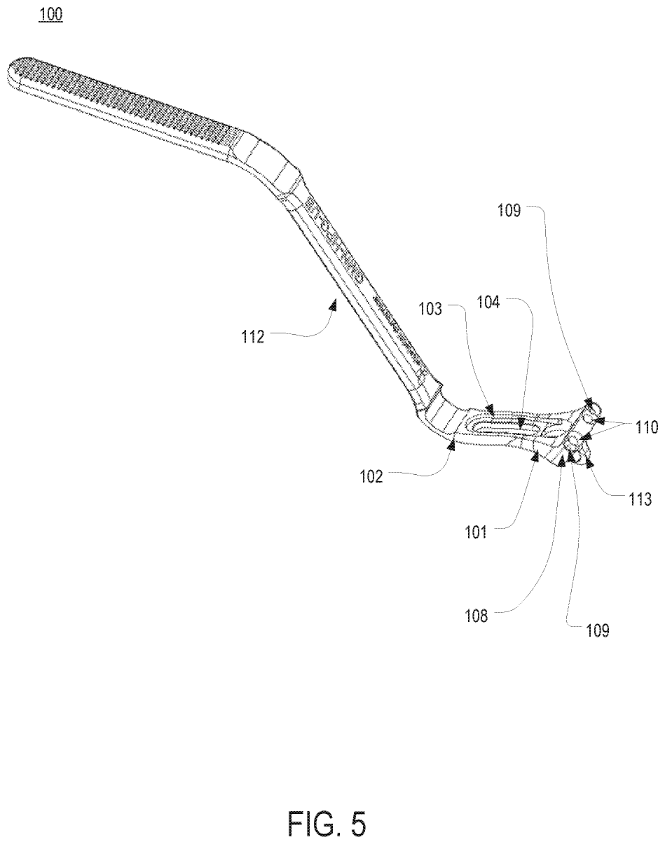

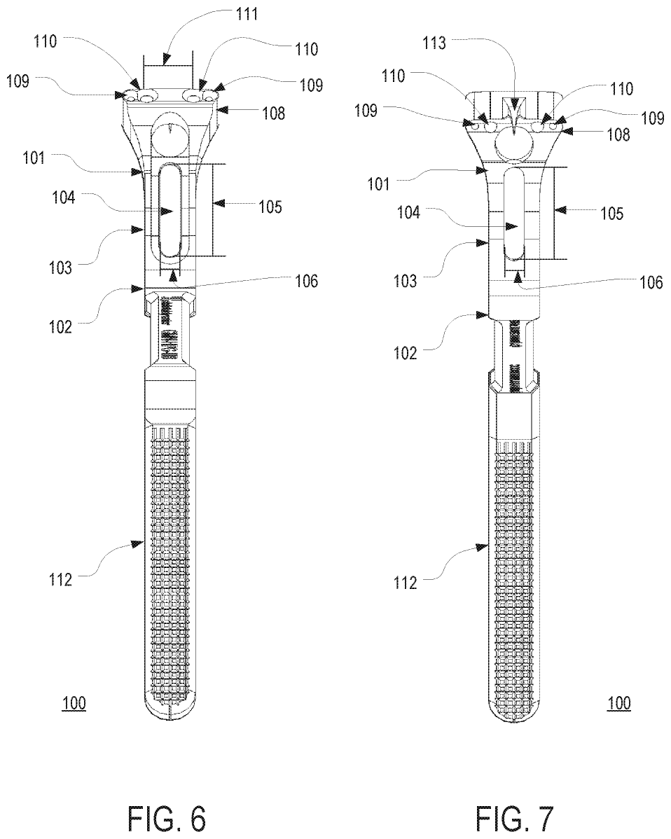

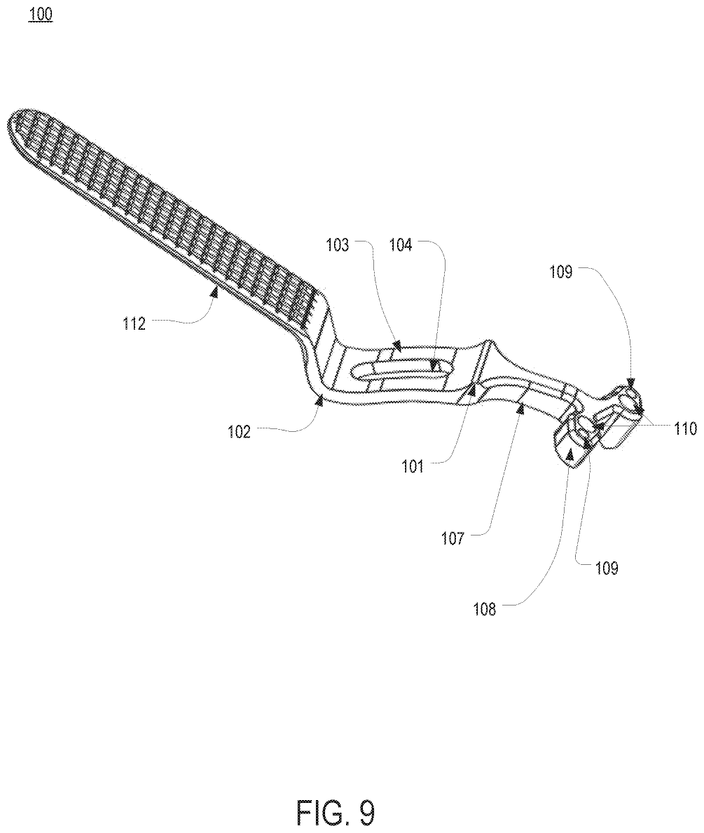

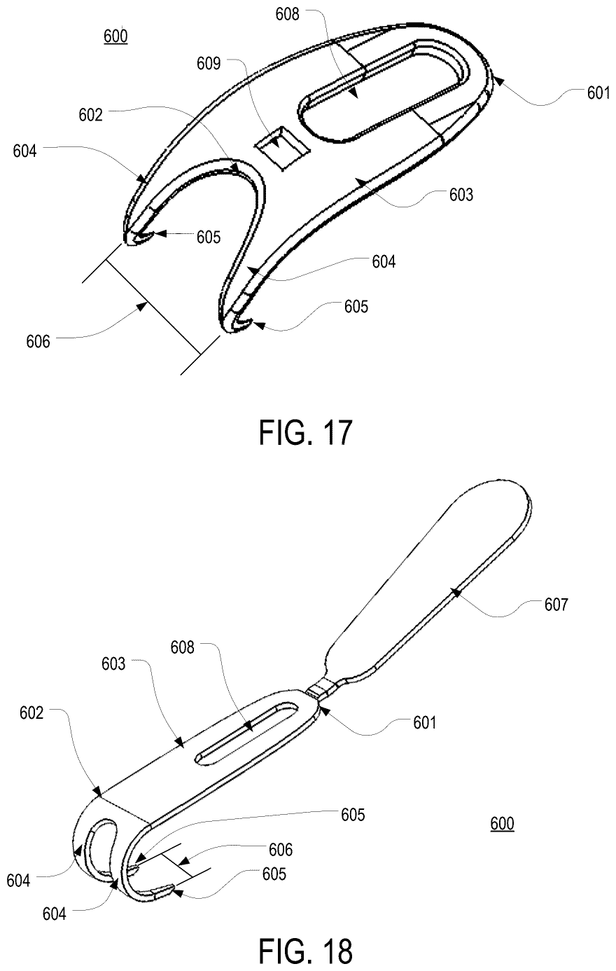

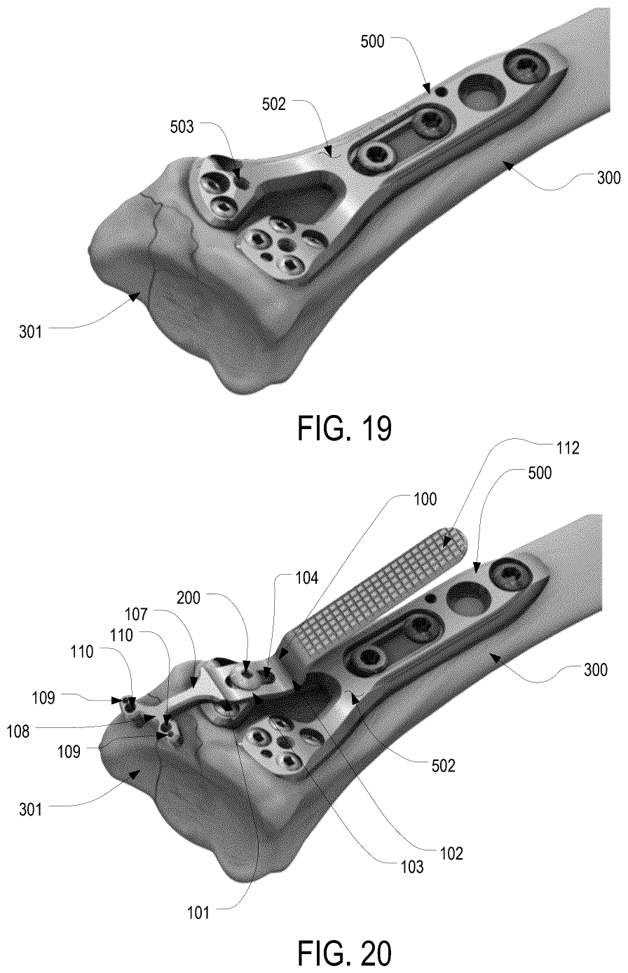

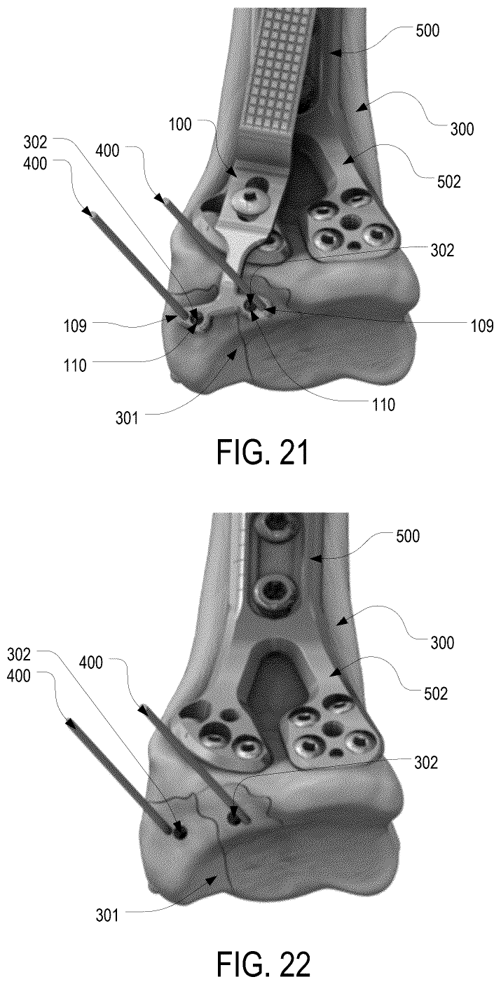

Referring first to FIGS. 1 through 4, shown are various views of a hook plate guide (100) for facilitating engagement of a hook plate (600) to a fracture fixation plate (500) and to at least one bone fragment (301) of a fractured bone (300) (see FIGS. 19 through 24) in accordance with one embodiment. The at least one bone fragment (301) may comprise at least one marginal fragment that is beyond reach of the fracture fixation plate (500) (see FIG. 19). The hook plate (600) may comprise at least one hook end (605) for securing the at least one bone fragment (301) (see FIGS. 17 and 18). In some embodiments, such as the ones depicted in the figures, the hook plate (600) may optionally comprise at least two hook ends (605) separated by a distance (606). Next, referring to FIGS. 5 through 8, another embodiment of the hook plate guide (100) is depicted. Referring now to FIGS. 9 through 12, shown are various views of yet another hook plate guide (100) in accordance with a further embodiment. FIGS. 20 and 21 illustrate one embodiment of the hook plate guide (100) engaged with a fracture fixation plate (500). Although the fracture fixation plate (500) is depicted throughout the figures as a volar fracture fixation plate, it should be understood that the hook plate guide (100) may be adapted to facilitate engagement of a variety of hook plates (600) to various fracture fixation plates (500) for different fractured bones (300).

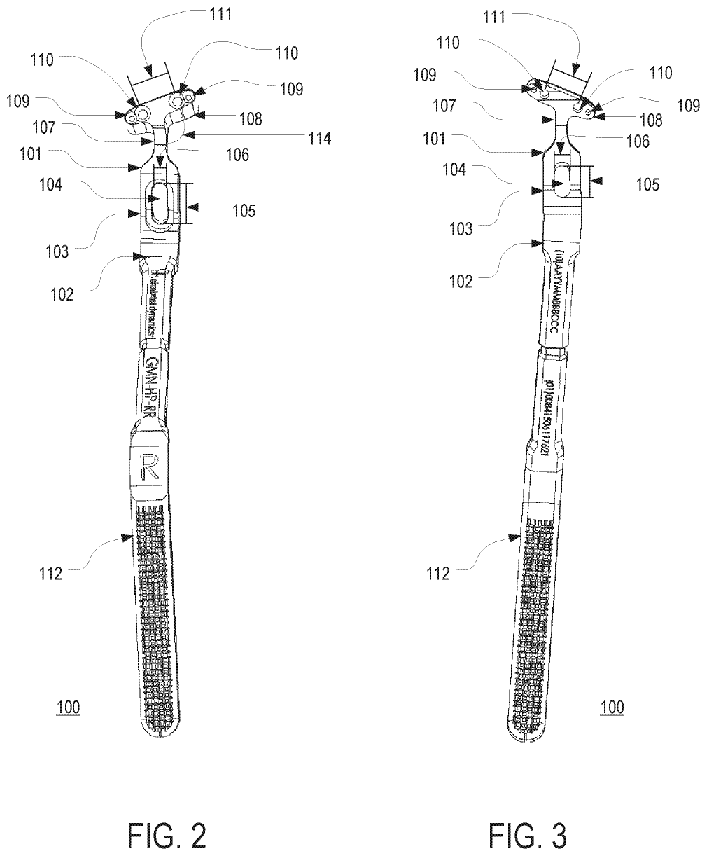

Now referring back to FIGS. 1-12, the hook plate guide (100) may comprise a distal end (101), a proximal end (102), and a substantially elongated rigid body (103) therebetween in accordance with one embodiment. As shown in FIGS. 1-3, 5-7 and 9-11, the body (103) may optionally comprise a slot (104). The slot (104) may be elongated and adapted to receive a hook plate guide fastener (200) (see FIG. 13). The slot (104) may be further adapted to slidably secure the hook plate guide (100) to the fracture fixation plate (500). Referring now to FIGS. 2-3, 6-7 and 10-11, the slot (104) of the hook plate guide (100) may comprise a length (105) and a width (106) and may be adapted to receive the hook plate guide fastener (200) at a selected position along the length (105) of the slot (104) in order to permit slidable securement of the hook plate guide (100) to the fraction fixation plate (500). In at least some embodiments, the hook plate guide fastener (200) may optionally be adapted to engage the fracture fixation plate to secure the hook plate guide (100). Shown in FIG. 20 is the slot (104) having received the hook plate guide fastener (200) to temporarily secure the hook plate guide (100) to the fracture fixation plate (500).

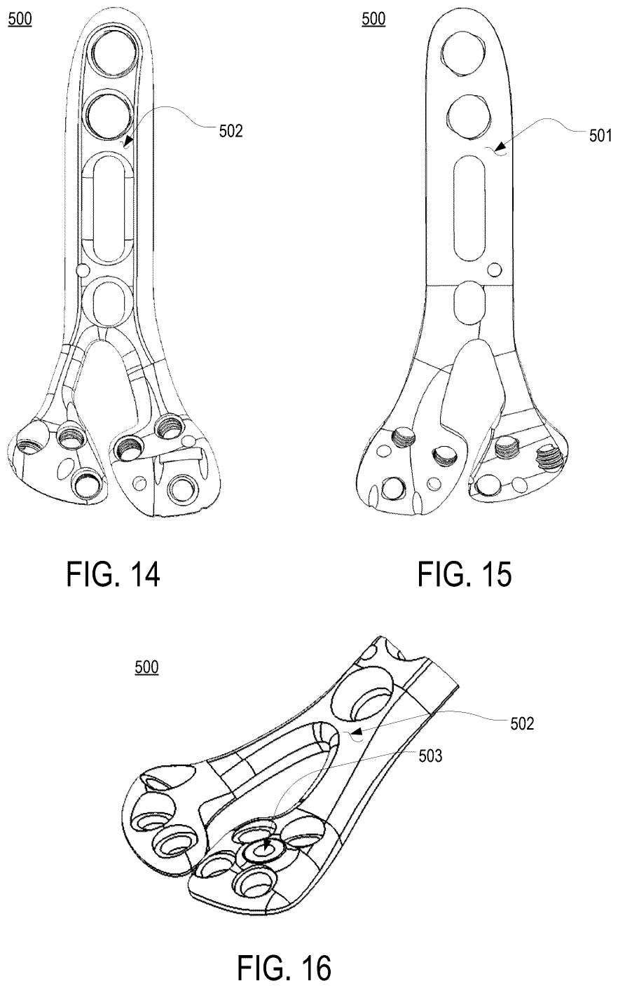

Referring next to FIGS. 14 through 16, shown are various views of an example fracture fixation plate (500) to which the hook plate guide (100) may facilitate engagement of a hook plate (600). The fracture fixation plate (500) may comprise a bone contacting surface (501) adapted to contact a fractured bone (300) and an opposite surface (502) on an opposite side of the bone contacting surface (501). The bone contacting surface (501) may be adapted to engage the fractured bone (300) (see FIG. 19), and the opposite surface (502) may be adapted to contact and be engaged by the hook plate guide (100) and/or the hook plate (600) (see FIGS. 20 and 24, respectively). Referring again to FIG. 16, the fracture fixation plate (500) may further comprise at least one hole (503). The hole (503) may optionally have an internal helical thread adapted to be engaged by a fastener, such as the hook plate guide fastener (200) or a hook plate fastener (700) (see FIGS. 19, 20 and 24).

Referring now to FIG. 13, an example hook plate guide fastener (200) is shown. The hook plate guide fastener (200) may comprise a head (201) having a diameter (202). As shown in FIG. 20, in at least one embodiment, the diameter (202) of the head (201) (see FIG. 13) may be larger than the width (106) of the hook plate guide (100) slot (104) (see FIGS. 2-3, 6-7 and 10-11) such that the head (201) is adapted to engage and secure the hook plate guide (100) when the hook plate guide fastener (200) is received by the slot (104) and engages the fracture fixation plate (500) (see FIG. 20). The larger diameter (202) of the head (201) compared to the slot (104) width (106) also prevents the hook plate guide fastener (200) from passing completely through the slot (104). The head (201) of the hook plate guide fastener (200) may further be adapted to receive a driving tool. The hook plate guide fastener (200) may also have an external helical thread (203) adapted to engage the internal helical thread of the hole (503) in the fracture fixation plate (500). In at least one embodiment, the hook plate guide fastener (200) (see FIG. 20) may be the same fastener as the hook plate fastener (700) subsequently used to secure the hook plate (600) to the fracture fixation plate (500) (see FIG. 24). Although the hook plate guide fastener (200) is illustrated as a screw, it should be understood that any suitable fastener may be used.

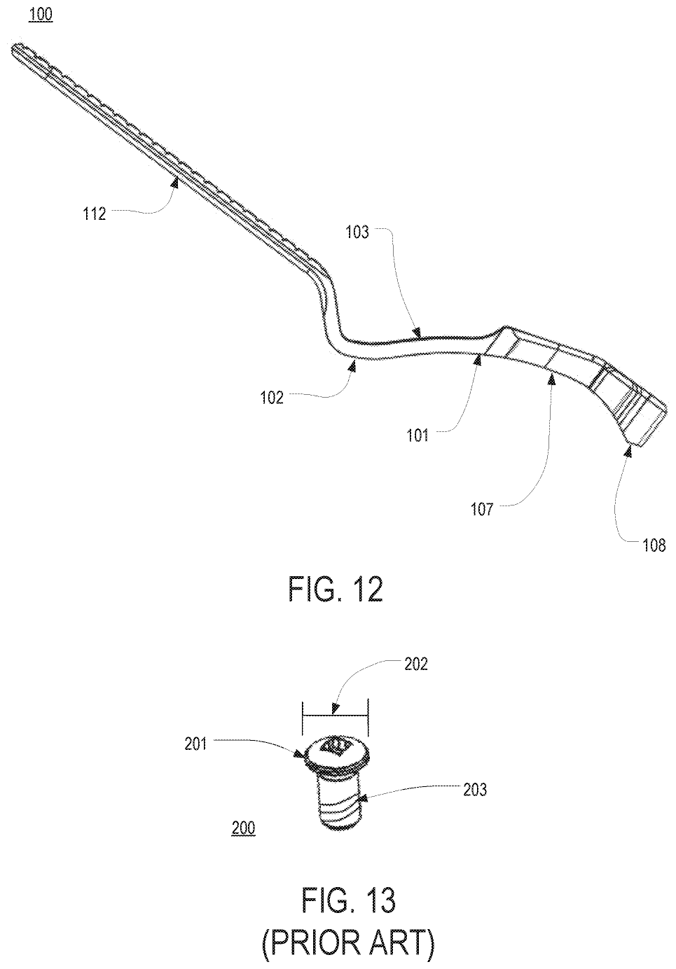

Referring now to FIGS. 1-3, 5-7 and 8-11, the distal end (101) of the hook plate guide (100) may optionally comprise a head (108). The head (108) may comprise at least one reference hole (109) and at least one drill hole (110). In at least some embodiments, such as the ones depicted in the figures, the head may optionally comprise at least two reference holes (109) and at least two drill holes (110) with a distance (111) between each of the at least two drill holes (110) (FIGS. 2-3, 6-7 and 10-11). The head (108) may extend outward. In at least one embodiment, the head (108) may optionally extend distally and curve downward to a position below the body (103) of the hook plate guide (100) (see, for example, FIG. 12). The at least one reference hole (109) and the at least one drill hole (110) may be adapted to be positioned over the at least one bone fragment (301) of the fractured bone (300) (see, for example. FIGS. 20 and 21). In at least some embodiments, each at least one drill hole (110) may be adjacent to a respective at least one reference hole (109). In some embodiments, a position of each at least one drill hole (110) may correspond to a position of a respective at least one hook end (605) of the hook plate (600). In embodiments having at least two drill holes (110), the distance (111) between each of the at least two drill holes (110) (see FIGS. 2-3, 6 and 10-11) may correspond to the distance (606) between each of the at least two hook ends (605) of the hook plate (600) (see FIGS. 17-18).

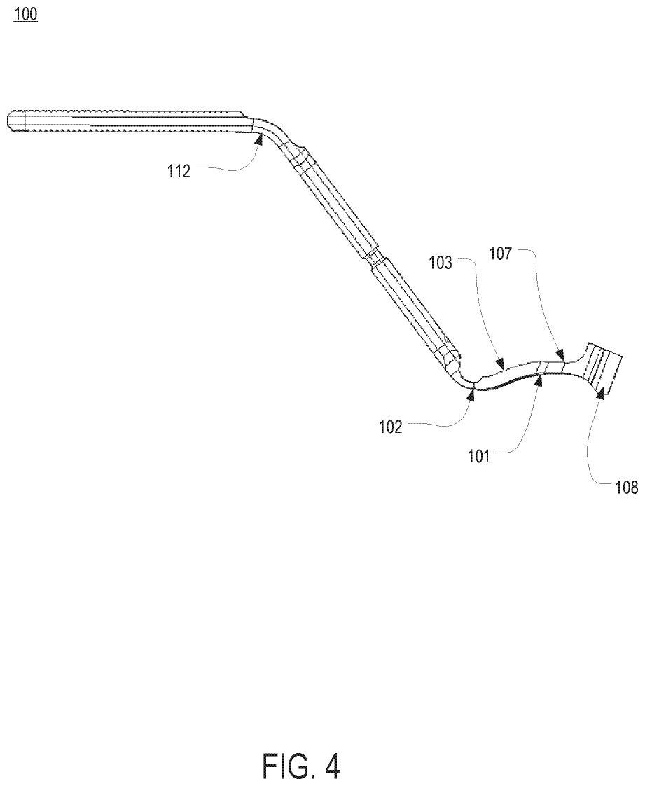

In at least some embodiments, such as the ones depicted in FIGS. 1-4, 9-12 and 20-21, the head (108) of the hook plate guide (100) may optionally be connected to the body (103) by an arm (107). The arm (107) may connect the head (108) to the distal end (101) of the hook plate guide (100). Referring to FIGS. 4, 12 and 20, in embodiments having an arm (107), the arm (107) may extend distally and curve downward to a position below the body (103) of the hook plate guide (100). In some embodiments having an arm (107), the arm (107) may be adapted to extend over the at least one bone fragment (301) such that the at least one reference hole (109) and the at least one drill hole (110) of the head (108) may be positioned over the at least one bone fragment (301). As shown in FIGS. 1-3 and 8-11, in at least some embodiments, the arm (107) may optionally be narrower than the body (103) of the hook plate guide (100). The arm (107) may also optionally be narrower than the head (108) of the hook plate guide (100).

It should be noted that a variety of different shaped arms (107) and heads (108) can be envisioned within the scope of the present apparatus and method of use. In additional embodiments, the head (108) of the hook plate guide (100) may optionally be thicker than the arm (107) and the body (103) (see, for example, FIG. 4).

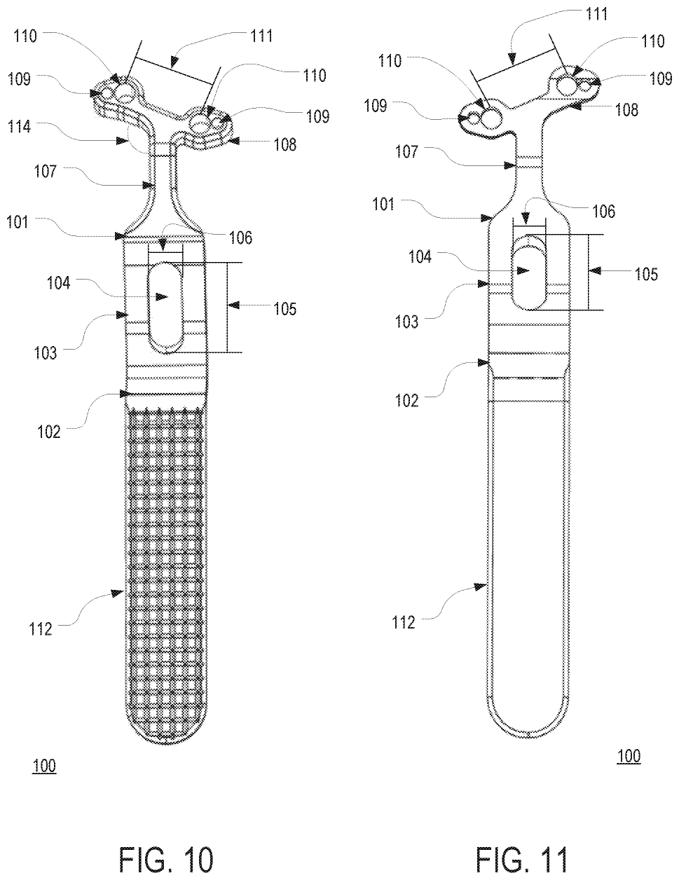

In some embodiments, the head (108) of the hook plate guide (100) may optionally be elongated and intersect the body (103) at an angle (114). Referring to FIGS. 2 and 10, in embodiments having an arm, the head (108) may be elongated and intersect the arm (107) at an angle (114). In at least some embodiments, such as the ones depicted in FIGS. 2 and 10, the angle (114) may be non-perpendicular. In other embodiments, the angle (114) may be perpendicular. In further embodiments, the angle (114) may optionally be adapted to correspond with the at least one bone fragment (301) of the fractured bone (300).

In at least one further embodiment, such as the one depicted in the hook plate guide (100) of FIGS. 5 through 8, the head (108) of the hook plate guide (100) may optionally further comprise a spike (113) adapted to engage the at least one bone fragment (301) (engagement not shown). The spike (113) may be a single point, a plurality of points, a plurality of teeth, or any other feasible means of engaging the at least one bone fragment (301).

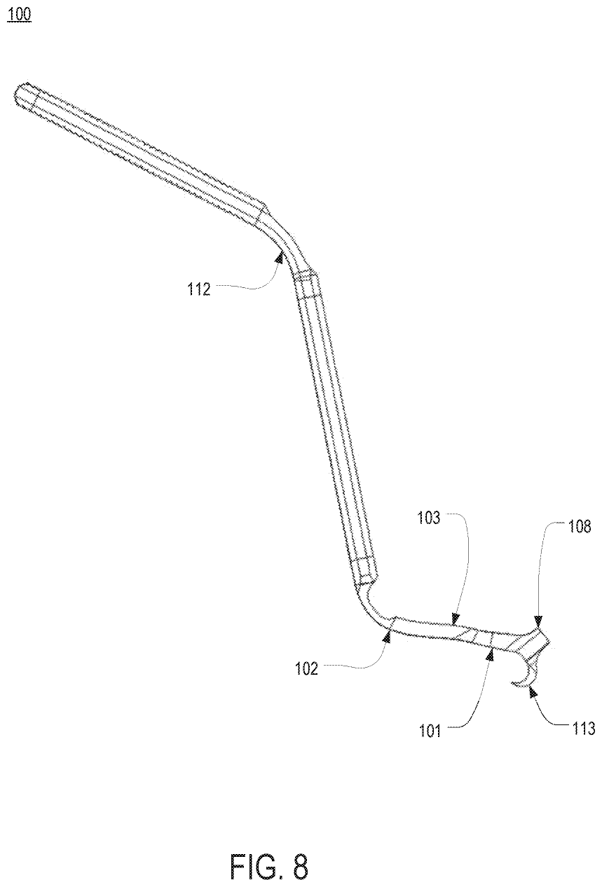

Referring next to FIGS. 1 through 12, the proximal end (102) of the hook plate guide (100) may optionally further comprise a grip member (112). The grip member (112) may extend proximally from the body (103) of the hook plate guide (100) to a position above the body (103). The grip member (112) may be adapted to facilitate in positioning the hook plate guide (100). Referring to FIGS. 4 and 8, in at least some embodiments, the grip member (112) may have a bend to allow for ease in maneuverability. In other embodiments, such as the one depicted in FIG. 9, the grip member (112) does not have a bend. In further embodiments, such as the embodiments depicted in FIGS. 1 through 12, the grip member (112) may optionally be textured to increase grip. The grip member (112) may be textured on one (FIGS. 10-11) or both (FIGS. 2-3 and 6-7) sides of the grip member (112).

Now referring to FIG. 21, each at least one reference hole (109) on the head (108) of the hook plate guide (100) may be adapted to receive a k-wire (400). In further embodiments, each at least one drill hole (110) may be adapted to receive a drill (not shown). Referring again to FIGS. 2-3, 6-7 and 10-11, in embodiments having at least two reference holes (109) and at least two drill holes (110), the at least two drill holes (110) may optionally be located interiorly with respect to the at least two reference holes (109).

Referring to FIGS. 17 and 18, shown are two examples of a hook plate (600) for which the hook plate guide (100) may facilitate engagement to the fracture fixation plate (500) and to at least one bone fragment (301). The hook plate (600) may comprise a proximal end (601), a distal end (602) with at least one projection (604) terminating in at least one hook end (605), and a body (603) therebetween. In at least some embodiments, such as the ones depicted, the hook plate (600) distal end (602) may comprise at least two projections (604) each terminating in a hook end (605), the hook plate (600) having at least two hook ends (605) separated by a distance (606). Referring to FIG. 24, the at least one projection (604) may be adapted to curve around the fractured bone (300) and the at least one bone fragment (301). The at least one hook end (605) may be adapted for insertion into a respective at least one bone fragment hole (302) formed using the hook plate guide (100) (see FIG. 24) to reduce the at least one bone fragment (301). Referring back to FIG. 18, the hook plate (600) proximal end (601) may optionally comprise an elongate member (607) extending proximally from the body (603) of the hook plate (600) to a position above the body (603) and is adapted to be pulled until a desired reduction of the at least one bone fragment (301) is achieved. The elongate member (607) may be removeable and adapted to break away from the hook plate (600). The hook plate (600) body (603) may be substantially flat and comprise a slot (608) adapted to receive a hook plate fastener (700) to secure the hook plate (600) to the fracture fixation plate (500) (see FIG. 24), thereby securing the at least one bone fragment (301) in a reduced position.

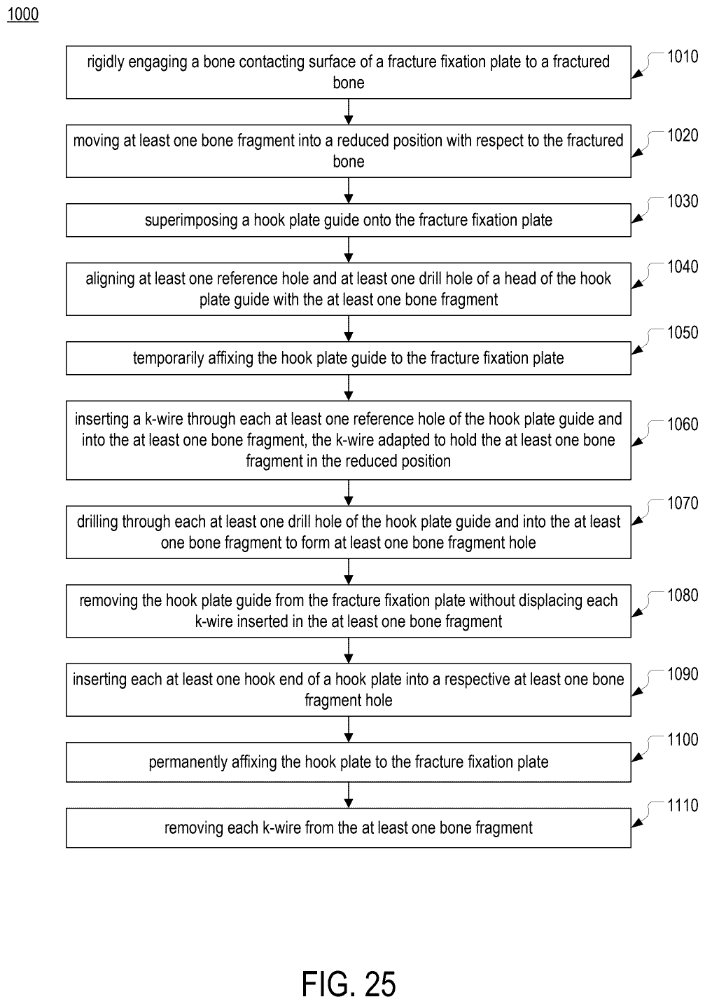

Now referring to FIG. 25, a sequence of steps is listed for a method (1000) of using a hook plate guide (100) to facilitate engagement of a hook plate (600) to a fracture fixation plate (500) and to at least one bone fragment (301) in a fractured bone (300) in accordance with one embodiment of the present method. In at least one embodiment, the fracture fixation plate (500) has a bone contacting surface, and the hook plate (600) has at least one hook end (605), as described previously (see FIGS. 14-16). In some embodiments, such as those depicted in the figures, the hook plate (600) may have at least two hook ends (605).

In one embodiment, the method (1000) comprises rigidly engaging the bone contacting surface (501) of the fracture fixation plate (500) to the fractured bone (300) (1010), as shown in FIG. 19. One or more fasteners may optionally be used to secure the fracture fixation plate (500) to the fractured bone (300).

Next, the at least one bone fragment (301) of the fractured bone (300) may be moved into a reduced position with respect to the fractured bone (300) (1020). The hook plate guide (100) may then be superimposed onto the fracture fixation plate (500) (1030), as shown in FIG. 20. Referring further to FIG. 20, and as described above, the hook plate guide (100) may comprise a distal end (101) having a head (108), a proximal end (102), and a substantially rigid body (103) therebetween. In some embodiments, the body (103) of the hook plate guide (100) may optionally further comprise an elongated slot (104) adapted to slidably secure the hook plate guide (100) to the fracture fixation plate (500) using a hook plate guide fastener (200). The head (108) may have at least one reference hole (109) and at least one drill hole (110) adapted to be positioned over the at least one bone fragment (301). In at least some embodiments, such as the ones depicted in the figures, the head may have at least two reference holes (109) and at least two drill holes (110).

Referring to FIG. 21, the at least one reference hole (109) and the at least one drill hole (110) may next be aligned with the at least one bone fragment (301) (1040). Optionally, the slot (104) on the body (103) of the hook plate guide (100) may be aligned with a hole (503) (see hole FIG. 19) on the fracture fixation plate (500). As shown in FIG. 21, in embodiments having at least two reference holes (109) and at least two drill holes (110), each of the at least two reference holes (109) may be aligned on either side of a fracture line of the at least one bone fragment (301). Similarly, in such embodiments, each of the at least two drill holes (110) may be aligned on either side of the fracture line of the at least one bone fragment (301).

Thereafter, the hook plate guide (100) may be temporarily affixed to the fracture fixation plate (500) (1050). FIG. 20 illustrates a hook plate guide (100) after it is temporarily affixed to the fracture fixation plate (500). The step of temporarily affixing the hook plate guide (100) may optionally be accomplished by inserting a hook plate guide fastener (200) through the slot (104) of the hook plate guide (100) and into a hole (503) on the fracture fixation plate (500), as shown in FIG. 20. While the hook plate guide fastener (200) is depicted as a screw, any fastener capable of securing the hook plate guide (100) to the fracture fixation plate (500) may be used. Optionally, the hook plate guide (100) may be temporarily affixed (see FIG. 20) to a location of the fracture fixation plate (500) that is the same as where the hook plate (600) is subsequently affixed (see FIG. 24).

In at least one further embodiment, the hook plate guide (100) may optionally comprise a spike (113) on the head (108) (see, for example, the hook plate guide (100) in FIGS. 5, 7 and 8). In such embodiments, the at least one bone fragment (301) may be engaged by the spike (113) on the hook plate guide (100) (not shown) prior to the step of temporarily affixing the hook plate guide (100) to the fracture fixation plate (500) (1050).

Now referring to FIG. 21, a k-wire (400) may then be inserted through each at least one reference hole (109) of the hook plate guide (100) and into the at least one bone fragment (301) (1060). The k-wire (400) may be adapted to hold the at least one bone fragment (301) in the reduced position. As previously described, in embodiments having at least two reference holes (109), each of the at least two reference holes (109) may be aligned on either side of the fracture line of the at least one bone fragment (301). Accordingly, in such embodiments, each k-wire (400) may be inserted on either side of the fracture line of the at least one bone fragment (301).

Next, referring again to FIG. 21, at least one bone fragment hole (302) is formed by drilling through each at least one drill hole (110) of the hook plate guide (100) and into the at least one bone fragment (301) (1070). In embodiments having at least two drill holes (110), at least two bone fragment holes (302) are formed when drilling through each drill hole (110). A k-wire may optionally be used for drilling. As previously described, in embodiments having at least two drill holes (110), each of the at least two drill holes (110) may be aligned on either side of the fracture line of the at least one bone fragment (301). Accordingly, in such embodiments, each of the at least two bone fragment holes (302) may be formed on either side of the fracture line of the at least one bone fragment (301).

Referring now to FIG. 22, the hook plate guide (100) may then be removed from the fracture fixation plate (500) without displacing each k-wire (400) inserted in the at least one bone fragment (301) (1080). This step may be accomplished by optionally disengaging a hook plate guide fastener (200) from a hole (503) on the fracture fixation plate (500).

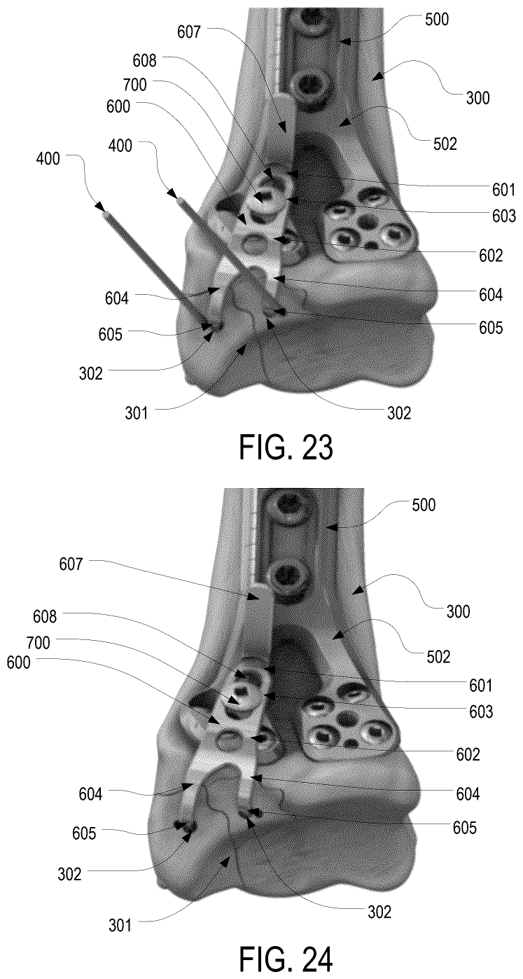

Thereafter, each at least one hook end (605) of the hook plate (600) may be inserted into a respective at least one bone fragment hole (302) (1090). FIG. 23 illustrates the hook plate (600) after it is affixed to the fracture fixation plate (500). In one embodiment, each k-wire (400) inserted in the at least one bone fragment (301) is optionally adapted to act as a guide to facilitate in locating the at least one bone fragment holes (302) for insertion of the at least one hook end (605). In embodiments having at least two drill holes (110), a distance (606) between each of at least two hook ends (605) of the hook plate is optionally equal to the distance (111) between each of the at least two drill holes (110) so that each of the at least two bone fragment holes (302) formed by drilling through the at least two drill holes (110) align with a respective one of the at least two hook ends (605).

Optionally, in embodiments where the hook plate (600) has an elongate member (607), as shown in FIGS. 23 and 24, after the at least one hook end (605) is inserted in the at least one bone fragment hole (302), the elongate member (607) may be pulled until a desired reduction of the at least one bone fragment (301) is achieved. In some embodiments, such as the one depicted in FIG. 17, the hook plate (600) may optionally comprise a tensioning port (609) located at a distal end (602) of the hook plate (600) and adapted to be pulled on by a tool (not shown) to achieve the desired reduction of the at least one bone fragment (301). In such embodiments, after the at least one hook end (605) is inserted in the at least one bone fragment hole (302), the tensioning port may be pulled on by the tool until the desired reduction of the at least one bone fragment (301) is achieved.

Referring back to FIG. 23, the hook plate (600) may then be permanently affixed to the fracture fixation plate (500) (1100). This step may optionally be accomplished by inserting a hook plate fastener (700) through a slot (608) on the hook plate (600) body (603) and into a hole (503) (see FIG. 19) of the fracture fixation plate (500), as shown in FIG. 24. In at least one embodiment, the hook plate fastener (700) and the hook plate guide fastener (200) may be the same fastener. Although the hook plate fastener (700) is depicted as a screw, any fastener capable of securing the hook plate (600) to the fracture fixation plate (500) may be used. In at least some embodiments, affixing the hook plate (600) to the fracture fixation plate (500) comprises securing the at least one bone fragment (301) in the reduced position with respect to the fractured bone (300). Optionally, the hook plate (600) may be affixed (see FIG. 24) to the same location on the fracture fixation plate (500) where the hook plate guide (100) was previously temporarily affixed (see FIG. 20).

After affixing the hook plate (600) to the fracture fixation plate (500), each k-wire (400) may be removed from the at least one bone fragment (301) (1110), as shown in FIG. 24. Once each k-wire (400) is removed, the hook plate (600) remains affixed to the fracture fixation plate (500) and the at least one bone fragment (301) remains in the reduced position with respect to the fractured bone (300).

In embodiments where the hook plate (600) has an elongate member (607), the elongate member (607) may optionally be removeable. In such embodiments, the elongate member (607) may be broken away and removed from the hook plate (600) (not shown) after permanently affixing the hook plate (600) to the fracture fixation plate (500).

Although described above in connection with particular embodiments, these descriptions are not intended to be limiting, as other guides can be made in accordance with the description herein, but of different size or scale, as desired. As such, although the apparatus and method of use are illustrated and described herein, various modifications and structural changes may be made therein without departing from the spirit and purview of the present application and within the scope and range of equivalents of the claims. Further, any steps described herein may be carried out in any desired order (and any additional steps may be added as desired and/or any steps may be deleted as desired). Encompassed embodiments of the present apparatus and method of use can be used in all plates calling for a guide.

Any reference in this specification to “one embodiment,” “an embodiment,” an “example embodiment,” etc., means that a particular feature, structure, or characteristic described in connection with the implementation is included in at least one embodiment of the present apparatus or method of use. The appearances of such phrases in various places in the specification are not necessarily referring to the same embodiment. In addition, any elements or limitations of any invention or embodiment thereof disclosed herein can be combined with any and/or all other elements or limitations (individually or in any combination) or any invention or embodiment thereof disclosed herein, and all such combinations are contemplated within the scope of the invention without limitation thereto.

As used herein, the terms “substantial” and “substantially” refer to the complete or nearly complete extent or degrees of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context, which one of ordinary skill in the art would be familiar with. However, generally speaking the nearness of completion will be so as to have the same overall result as if absolute or total completion were obtained.

The use of term “substantial” or “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

While a number of embodiments of the present apparatus and method of use have been described, it is understood that these examples and embodiments are illustrative only, and not restrictive, and that many modifications may become apparent to those of ordinary skill in the art and are to be included within the spirit and purview of this application. For example, any element described herein may be provided in any desired size (e.g., any element described herein may be provided in any desired custom size or any element described herein may be provided in any desired size selected from a “family” of sizes, such as small, medium, large). Further, one or more of the components may be made from many different suitable materials.

In addition, various modifications and additions can be made to the exemplary embodiments discussed without departing from the scope of the present apparatus and method of use. For example, while the embodiments described above refer to particular features, the scope and purview of this application also includes embodiments having different combinations of features and embodiments that do not include all of the described features. Accordingly, the scope of the present device and method of use is intended to embrace all such alternatives, modifications, and variations as within the scope of the claims, together with all equivalents thereof.

1. A hook plate guide for facilitating engagement of a hook plate to a fracture fixation plate and to at least one bone fragment of a fractured bone, the hook plate having at least one hook end for securing the at least one bone fragment, the guide comprising:
a distal end, a proximal end, and a substantially elongated rigid body therebetween;
the body comprising a slot, the slot being elongated and adapted to receive a hook plate guide fastener, the slot further adapted to slidably secure the hook plate guide to the fracture fixation plate;
the distal end comprising a head, the head having at least one reference hole and at least one drill hole, the head extending outward and the at least one reference hole and the at least one drill hole adapted to be positioned over the at least one bone fragment; and
wherein each at least one drill hole is adjacent to a respective at least one reference hole.

2. The hook plate guide of claim 1, wherein the head of hook plate comprises at least two reference holes and at least two drill holes, and a distance between each of the at least two drill holes corresponds to a distance between each of at least two hook ends of the hook plate.

3. The hook plate guide of claim 1, wherein a position of each at least one drill hole corresponds to a position of a respective at least one hook end of the hook plate.

4. The hook plate guide of claim 1, wherein the head is connected to the body by an arm.

5. The hook plate guide of claim 4, wherein the arm extends distally and curves downward to a position below the body of the hook plate guide.

6. The hook plate guide of claim 4, wherein the head is elongated and intersects the arm at an angle that is non-perpendicular.

7. The hook plate guide of claim 1, wherein the head is elongated and intersects the body at an angle that is non-perpendicular.

8. The hook plate guide of claim 1, wherein the head further comprises a spike adapted to engage the at least one bone fragment.

9. The hook plate guide of claim 1, wherein the proximal end further comprises a grip member extending proximally from the body to a position above the body, the grip member adapted to facilitate in positioning the hook plate guide.

10. The hook plate guide of claim 1, wherein each at least one reference hole is adapted to receive a k-wire.

11. The hook plate guide of claim 1, wherein each at least one drill hole is adapted to receive a drill.

12-15. (canceled)

16. The hook plate guide of claim 4, wherein the arm is narrower than the body of the hook plate guide.

17. The hook plate guide of claim 4, wherein the arm is narrower than the head of the hook plate guide.

18. The hook plate guide of claim 2, wherein the at least two drill holes are located interiorly with respect to the at least two reference holes.
